SEQUENCE LISTING (13 sequences)

SEQ ID NO: 1 - DNA, 20 bp, synthetic construct
  source 1..20: mol_type = other DNA
gatgctggtgctgagtatgt

SEQ ID NO: 2 - DNA, 20 bp, synthetic construct
  source 1..20: mol_type = other DNA
gcagaaggtgcagagatgat

SEQ ID NO: 3 - DNA, 23 bp, synthetic construct
  source 1..23: mol_type = other DNA
aggcagtgtaattagctgattga

SEQ ID NO: 4 - DNA, 20 bp, synthetic construct
  source 1..20: mol_type = other DNA
gtatcaacgcagagtacttt

SEQ ID NO: 5 - RNA, 22 nt, synthetic construct
  source 1..22: mol_type = other RNA
aggcagtgtaattagctgattg

SEQ ID NO: 6 - RNA, 22 nt, synthetic construct
  source 1..22: mol_type = other RNA
tttccgtcacattaatcgacta

SEQ ID NO: 7 - RNA, 20 nt, synthetic construct
  source 1..20: mol_type = other RNA
ttctccgaacgtgtcacgtg

SEQ ID NO: 8 - RNA, 20 nt, synthetic construct
  source 1..20: mol_type = other RNA
ttaagaggcttgcacagtgc

SEQ ID NO: 9 - RNA, 22 nt, synthetic construct
  source 1..22: mol_type = other RNA
caatcagctaattacactgcct

SEQ ID NO: 10 - RNA, 21 nt, synthetic construct
  source 1..21: mol_type = other RNA
cagtacttttgtgtagtacaa

SEQ ID NO: 11 - DNA, 22 bp, synthetic construct
  source 1..22: mol_type = other DNA
aggcagtgtaattagctgattg

SEQ ID NO: 12 - DNA, 269 bp, synthetic construct
  source 1..269: mol_type = other DNA
ggaataggaagacatcaagtgtcacttgaggcctagggcctgggattccttcgttaagaccagttgctcagggtggcgcaggaacaggacccaaccctgcgcccacttcctaccctctgcccctggcacagggccctgatctcagttgagccactgtcctacccgcatgccaacacacaatgtgccttctttctcaacagtgagcagtgctgggctactcctgacccaggcccggagggaactgaatcagtctttgaggtttttacctg

SEQ ID NO: 13 - DNA, 269 bp, synthetic construct
  source 1..269: mol_type = other DNA
ggaataggaagacatcaagtgtcacttgaggcctagggcctgggattccttcgttaagaccagttgctcagggtggcgcaggaacaggacccaaccctgcgcccacttcctaccctctgcccctggcacagggccctgatcagtcaactcggtgacaggatcccgcatgccaacacacaatgtgccttctttctcaacagtgagcagtgctgggctactcctgacccaggcccggagggaactgaatcagtctttgaggtttttacctg

(19) United States
(12) Patent Application Publication
(10) Pub. No.: US 2025/0163413 A1
(43) Pub. Date: May 22, 2025

(54) USE OF miR-34b IN REGULATING LIPID METABOLISM OF SHANDONG BLACK CATTLE

(71) Applicant: Qingdao Agricultural University, Qingdao (CN)

(72) Inventors: Yajuan DONG, Qingdao (CN); Ruili Liu, Qingdao (CN); Wenqi Zhang, Qingdao (CN); Xuejin Bo, Qingdao (CN); Zhenpeng Chen, Qingdao (CN)

(21) Appl. No.: 18/732,209

(22) Filed: Jun. 3, 2024

(30) Foreign Application Priority Data
Nov. 22, 2023 (CN) 202311562773.4

(51) Int. Cl. C12N 15/113; A01K 67/0278; C12N 15/85; C12Q 1/6883

(52) U.S. Cl. CPC C12N 15/113; A01K 67/0278; C12N 15/85; C12Q 1/6883; A01K 2217/072; A01K 2227/101; C12N 2310/141

(57) ABSTRACT

Use of a miR-34b in regulating lipid metabolism of a Shandong Black cattle is provided in the present disclosure, and belongs to the technical field of molecular biological breeding. A miR-34b can be used in breeding of the Shandong Black cattle to screen an individual with a low miR-34b level as a high-quality calf. Experiments have shown that the miR-34b can regulate proliferation and growth of an adipose tissue of the Shandong Black cattle, thereby regulating a birth weight of the calf. The miR-34b can affect proliferation and growth of adipocytes of the Shandong Black cattle, thereby affecting growth of fetal cattle. In breeding, the individual with a low miR-34b level should be selected as a high-quality calf of the Shandong Black cattle. Therefore, a novel direction is provided for breeding a high-quality beef Shandong Black cattle in the present disclosure.

### CROSS REFERENCE TO RELATED APPLICATION

This patent application claims the benefit and priority of Chinese Patent Application No. 202311562773.4 filed with the China National Intellectual Property Administration on Nov. 22, 2023, the disclosure of which is incorporated by reference herein in its entirety as part of the present application.

### REFERENCE TO SEQUENCE LISTING

This application incorporates-by-reference nucleotide sequences which are present in the file named “GWP20240402395_Sequence_Listing.xml”, which is 12,892 bytes in size, and which was created on May 20, 2024 in the IBM-PC machine format, having an operating system compatibility with MS-Windows, which is contained in the xml file filed Jun. 3, 2024 as part of this application.

### TECHNICAL FIELD

The present disclosure belongs to the technical field of molecular biological breeding, and specifically relates to use of a miR-34b in regulating lipid metabolism of a Shandong Black cattle.

### BACKGROUND

Shandong Black cattle is the first novel beef cattle germplasm successfully bred through somatic cell nuclear transfer in China. By means of cross-breeding and molecular marker-assisted breeding, it has overcome the shortcomings of Luxi Yellow and Bohai Black, by the semen from a stud bull of Japanese Black, the Luxi Yellow is improved such that its offspring can show a combination of excellent traits. The Shandong Black cattle has a tall body, well-developed muscles, obvious tendons, thin skin and fine bones, a strong constitution, and a well-proportioned structure. The beef from this breed is tender and juicy, shows a desirable taste, and is also called “snowflake beef” due to a typical marble pattern. Moreover, the beef is rich in protein with an amino acid composition closer to the demands of human body than that of pork, and can improve the body's disease resistance capability, with the function of nourishing the heart muscle, and enhancing immunity. Meanwhile, the beef is rich in iron, has very low saturated fatty acid content in intermuscular fat and high unsaturated fatty acid content, and exhibits a unique flavor. In 2015, this breed was recognized by experts as a novel breed that inherited the specialized beef breed characteristics of Japanese Black and is an excellent breed of cattle for producing marbled beef.

Many researchers have concluded that the birth weight of a calf is an indispensable part of the target traits of cattle breeding and an important factor affecting the production performance of beef cattle. The birth weight of a calf is the first and most important growth trait measured after birth. As an important component of the birth weight of livestock and poultry, adipose tissue is also an important factor affecting their meat production performance. During breeding of meat-producing animals, studies have revealed that, a genetic mechanism affecting the intramuscular fat (IMF) content can pose certain guiding significance for improving the growth and development of livestock and poultry and improving meat production performance, thereby further enabling the beef industry to improve selective breeding standards and meat value.

In animals, systemic control of metabolism is completed by metabolic tissues and relies on the regulatory cycling of a plethora of molecules, such as hormones and lipoprotein complexes. MicroRNAs (miRNAs), a family of post-transcriptional gene repressors, are present throughout the animal kingdom and are widely implicated in the regulation of gene expression in a variety of environments, including nearly all aspects of metabolic system control. The miRNAs are small non-coding single-stranded RNAs with a length of about 22 nucleotides. There are currently nearly 40,000 types of miRNAs discovered. The miRNAs are widely present in a variety of eukaryotes and play a broad regulatory role in biological processes such as cell proliferation, differentiation, and apoptosis, thus exerting a highly important influence on the growth and development of organisms. miR-34b is a member of the miR-34 family (including miR-34a, miR-34b, and miR-34c) and has a mature sequence of 23 nucleotides. The miR-34 family is a well-known cancer inhibitor that can be induced by p53 and participates in the biological processes of many cancers. It has been reported that miR-34b-5P in chickens can promote the proliferation of avian leukosis virus subgroup J (ALV-J)-infected cells by targeting anti-melanoma differentiation-associated gene 5 (MDA5). In addition to cancer, miR-34 is also differentially expressed during skeletal muscle development in Nile tilapia. The miR-34a can also promote the proliferation of human pulmonary artery smooth muscle cells. In addition, the miR-34a and miR-34c can inhibit the proliferation of smooth muscle cells. All members of the miR-34 family may participate in and influence preadipocyte differentiation and postnatal fat gain during mouse embryonic development. According to another research report, the miR-34b controls milk fat biosynthesis through an AKT/MTER signaling pathway. All these studies indicate that the miR-34b can not only regulate the physiological processes of cancer but also participate in regulating fat biosynthesis during certain physiological activities.

Sphingosine 1 phosphate lyase 1 (SGPL1), as a key enzyme during the sphingolipid metabolism, is the only intracellular enzyme that catabolizes SIP. The SGPL1 affects the metabolic homeostasis of sphingolipids by directly regulating SIP levels in vivo. Studies by BEKTAS et al. have shown that compared with normal mice, the levels of sphingolipid intermediate metabolites sphingosine, dihydrosphingosine, and ceramide are increased in the serum and liver of SGPL1-knockout mice, while the levels of phospholipids, TG, diacylglycerol, and cholesterol are also increased in non-sphingolipid metabolic pathways. This indicates that SGPL1 not only regulates sphingolipid metabolism, but also affects the metabolism of other lipids. Further studies have shown that the SGPL1 mutation causes the upregulation of the expression of both fat synthesis and decomposition and oxidation genes in adipose tissue, but with a predominance of the decomposition and oxidation genes. In addition, the SGPL1 promotes the proliferation and migration of lung adenocarcinoma cells, and promotes the upregulation of the expression of the lipolysis rate-limiting enzyme HSL and fatty acid oxidation key enzyme CPT1 as well as LPL and PPAR-γ. Taken together, it is shown that the SGPL1 plays an important role in lipid and fat metabolism. Overexpression of the SGPL1 can cause serum lipid metabolism disorders, enhanced fat oxidation and decomposition, and reduced fat content in animals, which has potential clinical significance and provides new research ideas and regulatory targets for regulating lipid metabolism.

However, both miR-34 and SGPL1 still show an unclear function in the growth and development of beef cattle, and there are no relevant reports, such that their functions on the birth weight of calves require further confirmation in order to improve and continue the excellent quality of Shandong Black cattle.

### SUMMARY

In order to solve the problems existing in the prior art, the present disclosure provides use of a miR-34b in regulating the expression of proliferation and differentiation of adipocytes in a Shandong Black cattle, which promotes proliferation and growth of an adipose tissue and thus affecting the birth weight of a calf.

To achieve the above objective, the present disclosure adopts the following technical solutions:

The present disclosure provides use of a miR-34b in breeding a Shandong Black cattle; where the miR-34b has a nucleotide sequence set forth in SEQ ID NO: 11.

In the present disclosure, the use refers to screening an individual with a low miR-34b level as a stud bull.

The present disclosure provides use of a miR-34b in regulating adipose proliferation and differentiation in a Shandong Black cattle.

In some embodiments, the use refers to transfecting a target cell with a vector containing a mimic or an inhibitor of the miR-34b.

In some embodiments, the mimic of the miR-34b has a nucleotide sequence set forth in SEQ ID NO: 5 or SEQ ID NO: 6; and the inhibitor of the miR-34b has a nucleotide sequence set forth in SEQ ID NO: 9.

In some embodiments, the vector includes but is not limited to a lentivirus, an adenovirus, an adeno-associated virus (AAV), a liposome, and a plasmid for constructing a virus.

In some embodiments, the target cell is selected from the group consisting of a somatic cell, a fertilized ovum, and a cell line that are derived from the Shandong Black cattle.

The present disclosure has the following advantages:

Experiments have shown that the miR-34b can regulate proliferation and growth of an adipose tissue of the Shandong Black cattle, thereby regulating the birth weight of the calf. The miR-34b can affect proliferation and growth of adipocytes of the Shandong Black cattle, thereby affecting growth of a fetal cattle. In breeding, the individual with a low miR-34b level should be selected as a high-quality calf of the Shandong Black cattle. Therefore, a novel direction is provided for breeding a high-quality beef Shandong Black cattle in the present disclosure.

### BRIEF DESCRIPTION OF THE DRAWINGS

FIG. 1A-B show the cluster analysis diagram and differential expression diagram of differential miRNAs;

FIG. 2A-B show the analysis of miR-34b expression levels in different tissues;

FIG. 3 shows the culture process of preadipocytes after miR-34b transfection (24 h and 48 h);

FIG. 4 shows the proliferation rates of bovine adipocytes at different time points;

FIG. 5 shows the morphology of the preadipocytes at different time points of induced differentiation;

FIG. 6A-C show the screening and enrichment analysis diagram of miR-34b target gene;

FIG. 7A-B show the detection results of an interaction between bta-miR-34b and SGPL1-3′UTR; and

FIG. 8A-B show the expression level of SGPL1 detected by fluorescence quantification and immunofluorescence.

### DETAILED DESCRIPTION OF THE EMBODIMENTS

The present disclosure is further described in the following with reference to the specific examples, but the present disclosure is not limited by the following examples.

### Example 1 Screening and Expression Identification of Differential miRNAs

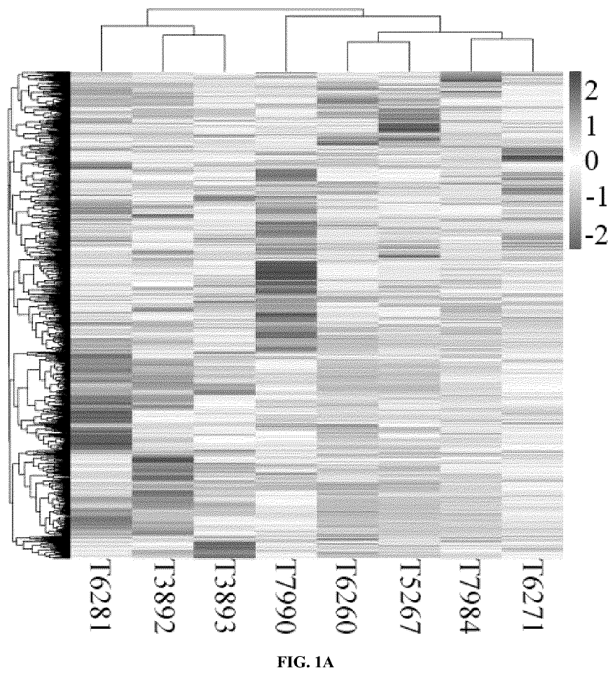

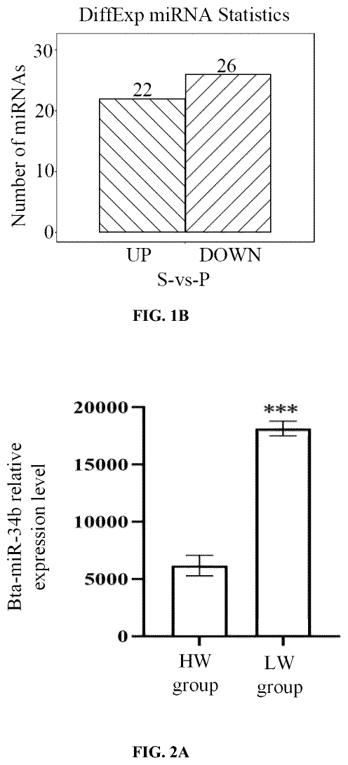

Shandong Black cattle were selected from Shandong Zhaofu Animal Husbandry Co., Ltd. In the experiment, 8 healthy Shandong Black cattle waiting to give birth were selected and raised in the same gestation house with desirable lighting and ventilation, appropriate house temperature and humidity, consistent feeding ration and drinking water. Phenotypic data such as birth weight, placental weight and size were measured immediately after the birth of the fetal cattle, and the fetal cattle were divided into 4 low-birth weight groups (LW group, 28.5 kg≤birth weight≤31.5 kg) and 4 high-birth weight groups (HW group, 32 kg≤birth weight ≤36 kg) based on their birth weights. Their placenta tissues were separately collected and stored in liquid nitrogen. Total RNA was extracted using Trizol reagent (Invitrogen) separately. The extracted RNA samples with desirable integrity and high purity were selected to construct a sequence library: after sequencing using an Illumina Genome Analyzer system, an sRNA library was constructed. The clean reads from sRNA sequencing were mapped to the reference genome through the alignment analysis software Bowtie (setting to allow one mismatch), and a total of 15,421,902 clean reads were obtained; compared with the reference sequence of bovine species from the miRBase database, there were 677 clean reads in total matched to known miRNAs (FIG. 1A). According to the differential miRNA screening criteria: |log 2 (Fold_change)|≥1 and Q≤0.05, 48 differentially-expressed miRNAs were obtained (Table 1), of which 22 were up-regulated and 26 were down-regulated (FIG. 1B). According to Transcripts Per Million (TPM), the differentially-expressed miRNAs in Shandong Black cattle were selected as miR-34b.

TABLE 1
Differentially-expressed miRNAs

| | miRNA name |
|---|---|
| Down- | bta-miR-2284z, bta-miR-2285b. bta-miR-2285bm, bta-miR-2425-5p, bta-miR-2484, |
| regulation | bta-miR-34b, bta-miR-383, miR-11988-z, miR-16-z, miR-183-x, miR-186-y, miR-276-y, |
| | miR-378-x, miR-664-x, miR-7262-y, novel-m0025-3p, novel-m0049-3p. novel-m0054-5p, |
| | novel-m0058-5p, novel-m0059-5p, novel-m0060-5p, novel-m0061-5p, novel-m0089-5p, |
| | novel-m0130-3p, novel-m0200-3p, novel-m0201-5p |
| up- | bta-miR-124a. bta-miR-124b, bta-miR-125b, bta-miR-153, bta-miR-196a, bta-miR-217, |
| regulation | bta-miR-2285bi, bta-miR-2355-5p, miR-12030-z, miR-125-z, miR-134-y, miR-15-z, |
| | miR-324-x, miR-4301-z, miR-496-x, miR-5100-z, miR-615-y, miR-6596-x, miR-665-x, |
| | miR-9027-z, novel-m0094-3p, novel-m0112-3p |

The placental tissues of the HW group and the LW group (4 animals in each group) were selected, while muscles, hearts, kidneys, livers, spleens, and adipose tissues of 2-month-old Shandong Black cattle (3) were collected and stored in liquid nitrogen. The total RNA was extracted with Trizol reagent (Invitrogen), respectively, and a cDNA was obtained by reverse transcription. The cDNA was used as a template to conduct SYBR Green qPCR on a miR-34b gene using glyceraldehyde-3-phosphate dehydrogenase (GAPDH) as a reference gene. A CT value of each sample was detected and averaged, and relative expression levels of the miR-34b in different groups and tissues were calculated.

TABLE 2
qPCR primers for each gene

| Gene name | Primer | Sequence 5′-3′ |
|---|---|---|
| GAPDH | Upstream | GATGCTGGTGCTGAGTATGT |
| | | (SEQ ID NO: 1) |
| GAPDH | Downstream | GCAGAAGGTGCAGAGATGAT |
| | | (SEQ ID NO: 2) |
| miR-34b | Upstream | AGGCAGTGTAATTAGCTGATTGA |
| | | (SEQ ID NO: 3) |
| miR-34b | Downstream | GTATCAACGCAGAGTACTTT |
| | | (SEQ ID NO: 4) |

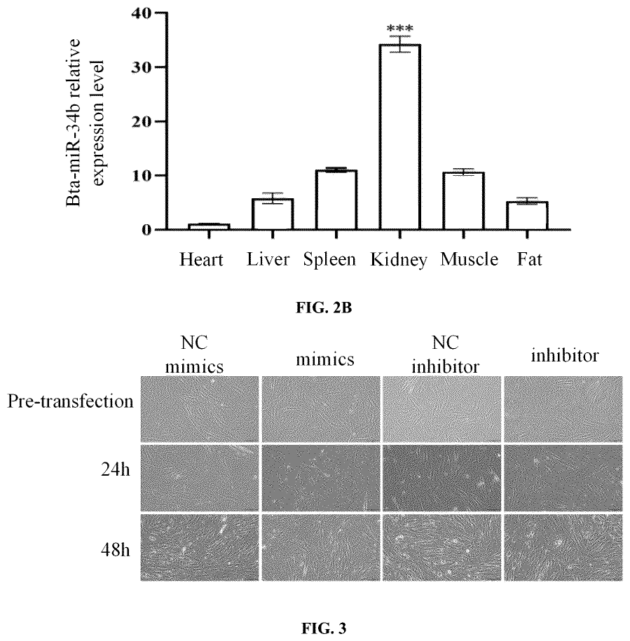

The results of fluorescence quantitative PCR showed that the expression level of miR-34b was low in the HW group but high in the LW group (FIG. 2A). Meanwhile, the expression profile of each tissue showed that the expression level of miR-34b was the highest in the kidney, which was extremely significantly higher than that in other tissues (P<0.01), while the lowest expression levels appeared in spleen, muscle, liver, fat, and heart in sequence (FIG. 2B).

### Example 2 Influence of miR-34b on Differentiation and Proliferation of Adipocytes in Shandong Black Cattle

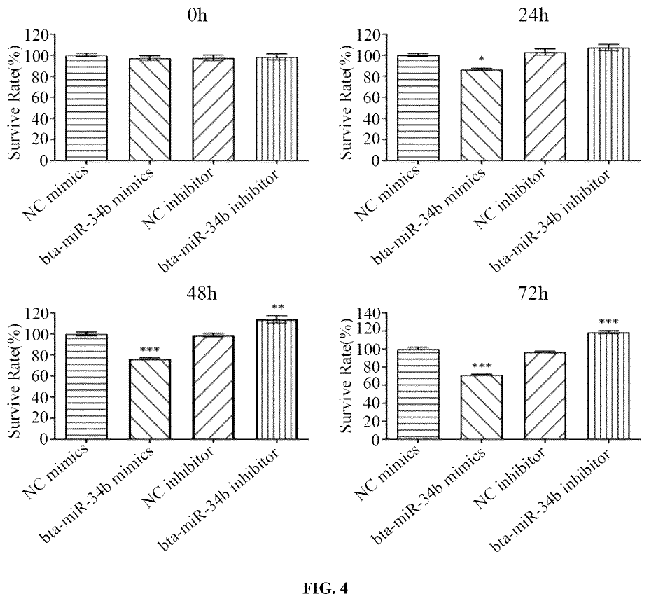

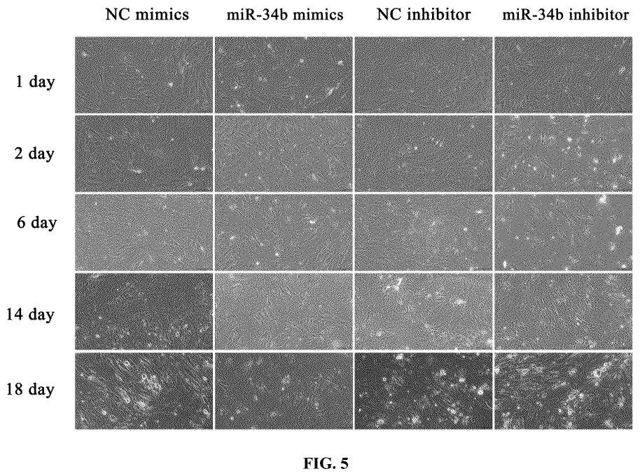

Bovine primary cells were isolated and cultured from the hindlimb fat of newborn Shandong Black cattle calves. The cells were collected after reaching 80% confluence, and the concentration of a cell suspension was adjusted with complete medium. The cells were divided into 6-well plates, with 2×10⁵ cells/well, 2 mL per well, and cultured in a 37° C., 5% CO₂ incubator for 24 h. The cells were transfected with miR-34b overexpression negative control (NC mimic), miR-34b overexpression (miR-34b mimic), miR-34b inhibition negative control (NC inhibitor), and miR-34b inhibition (miR-34b inhibitor). After culturing for another 48 h, the medium was changed to a high-glucose DMEM medium with 2% horse serum to continue the culturing. The morphology of the cells was observed 24 h and 48 h separately post-transfection (FIG. 3). A cell proliferation rate was detected by CCK-8 method at 0 h, 24 h, 48 h, and 72 h separately (FIG. 4).

TABLE 3
Mimic and inhibitor sequences of miR-34b

| Gene name | Sequence |
|---|---|
| mimic | 5′-3′AGGCAGUGUAAUUAGCUGAUUG |
| | (SEQ ID NO: 5) |
| | 3′-5′ AUCAGCUAAUUACACUGCCUUU |
| | (SEQ ID NO: 6) |
| NC mimic | 5′-3′ UUCUCCGAACGUGUCACGUG |
| | (SEQ ID NO: 7) |
| | 3′-5′ CGUGACACGUUCGGAGAAUU |
| | (SEQ ID NO: 8) |
| inhibitor | 5′-3′ CAAUCAGCUAAUUACACUGCCU |
| | (SEQ ID NO: 9) |
| NC inhibitor | 5′-3′ CAGUACUUUUGUGUAGUACAA |
| | (SEQ ID NO: 10) |

TABLE 4
Cell proliferation rate detected by CCK-8 method

| Transfection plasmid | 0 h | 24 h | 48 h | 72 h |
|---|---|---|---|---|
| miR-34b mimic | 0.5035 | 0.5714 | 0.6512 | 0.7633 |
| miR-34b mimic-NC | 0.5175 | 0.6622 | 0.8512 | 1.0713 |
| miR-34b inhibitor | 0.5090 | 0.7108 | 0.9699 | 1.2711 |
| miR-34b inhibitor-NC | 0.5040 | 0.6821 | 0.8426 | 1.0348 |

The results of cell proliferation rate detected by CCK-8 method were shown in Table 4: there was no difference among the treatment groups at 0 h; at 24 h, compared with the NC group, the miR-34b mimic began to reduce the cell proliferation rate (P<0.05), while the miR-34b inhibitor group increased the proliferation rate; at 48 h and 72 h, the miR-34b mimic group significantly reduced the cell proliferation rate (P<0.01), while the inhibitor group significantly increased the cell proliferation rate (P<0.01). In summary, the results of CCK-8 and EdU methods both indicated that the miR-34b inhibited the proliferation of adipocytes in Shandong Black cattle.

The morphology of cells of different treatments 2 d post-transfection was shown in FIG. 5: cell fusion began to appear after 2 d of induced differentiation using a universal adipogenic induction medium; compared with the NC group, there were less cell fusions in the miR-34b mimic group, while a large number of cells in the miR-34b inhibitor group showed fusion, and their morphology gradually changed from spindle-shaped to oval-shaped; on Day 3, compared with the NC group, small lipid droplets began to appear in the cytoplasm of the miR-34b inhibitor group, and a small number of lipid droplets appeared in the miR-34b mimic group, with no significant difference in number; after continuing to culture for 6 d, it was observed that the number of lipid droplets in the cells increased, and the lipid droplets gradually became larger in size and fused; at 14-16 d of differentiation, lipid droplets increased significantly in the miR-34b inhibitor group and decreased significantly in the miR-34b mimic group, indicating that the cells had differentiated into mature adipocytes. This indicated that the miR-34b could inhibit bovine adipocyte differentiation.

### Example 3 Screening and Expression Identification of miR-34b Target Gene

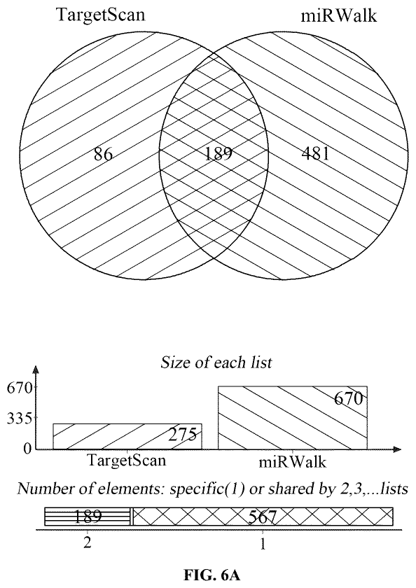

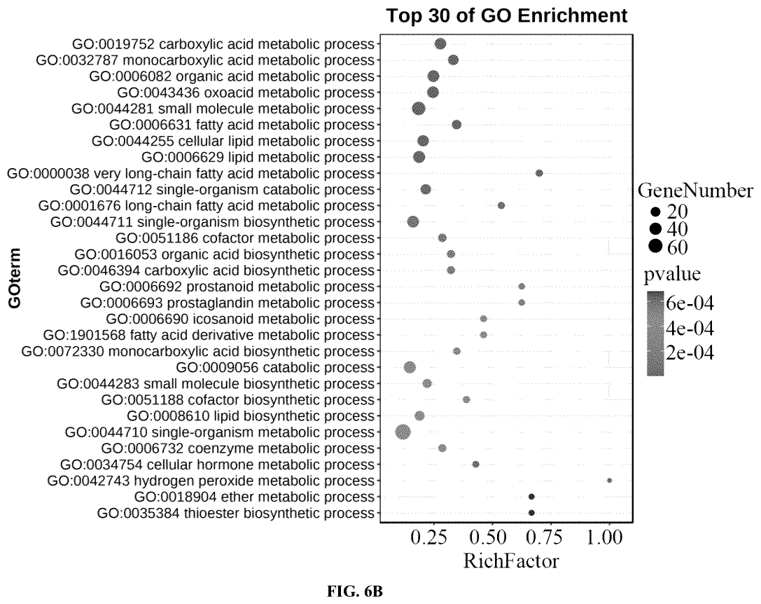

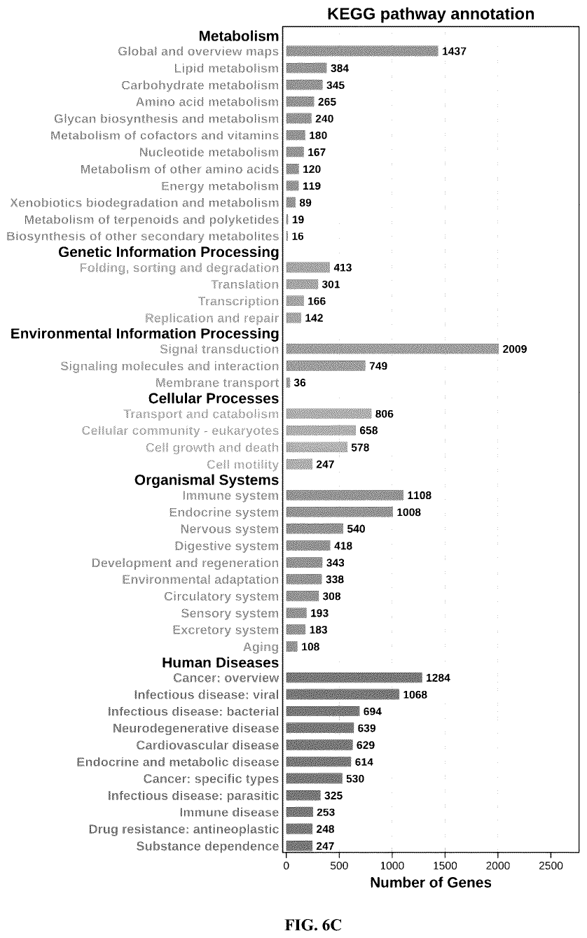

TargetScan 7.2 software was used to predict target genes for bta-miR-34b, and a total of 189 target genes were predicted (FIG. 6A). GO functional annotation (FIG. 6B) showed that the miR-34b was significantly enriched in cellular functional entries such as carboxylic acid metabolism and organic acid metabolism process. KEGG enrichment (FIG. 6C) showed that the miR-34b target gene was significantly enriched in pathways related to lipid metabolism, carbohydrate metabolism, and amniotic fluid acid metabolism. This indicated that the miR-34b was involved in regulating processes related to lipid metabolism. Based on the GO and KEGG enrichment results, SGPL1 was finally selected as its target gene and subsequent functional verification was conducted.

### Example 4 Gene Expression Control of miR-34b and SGPL1

A gene in 3′ non-coding region of the SGPL1 gene was synthesized according to Table 5 separately, where WT represented a wild type and MUT represented a mutant type. Enzyme digestion sites were added onto both ends of the gene, and a pSI-Check2 vector which was also double-enzymatically digested was ligated to the gene after double-enzymatic digestion. The plasmids with correct sequence after verification were named SGPL1-3′UTR-WT and SGPL1-3′UTR-MUT, respectively.

TABLE 5
Sequences of 3′ non-coding region of miR-34b and SGPL1 genes
(underlined part indicating mutation sites)

| Gene name | Sequence 5′-3′ |
|---|---|
| bta-miR-34b | AGGCAGTGTAATTAGCTGATTG (SEQ ID NO: 11) |
| SGPL1-3UTR-WT | GGAATAGGAAGACATCAAGTGTCACTTGAGGCCTAGGGCCTG |
| | GGATTCCTTCGTTAAGACCAGTTGCTCAGGGTGGCGCAGGAA |
| | CAGGACCCAACCCTGCGCCCACTTCCTACCCTCTGCCCCTGG |
| | CACAGGGCCCTGATCTCAGTTGAGCCACTGTCCTACCCGCA |
| | TGCCAACACACAATGTGCCTTCTTTCTCAACAGTGAGCAGTG |
| | CTGGGCTACTCCTGACCCAGGCCCGGAGGGAACTGAATCAGT |
| | CTTTGAGGTTTTTACCTG (SEQ ID NO: 12) |
| SGPL1-3UTR-MUT | GGAATAGGAAGACATCAAGTGTCACTTGAGGCCTAGGGCCTG |
| | GGATTCCTTCGTTAAGACCAGTTGCTCAGGGTGGCGCAGGAA |
| | CAGGACCCAACCCTGCGCCCACTTCCTACCCTCTGCCCCTGG |
| | CACAGGGCCCTGATCAGTCAACTCGGTGACAGGATCCCGCA |
| | TGCCAACACACAATGTGCCTTCTTTCTCAACAGTGAGCAGTG |
| | CTGGGCTACTCCTGACCCAGGCCCGGAGGGAACTGAATCAGT |
| | CTTTGAGGTTTTTACCTG (SEQ ID NO: 13) |

The bta-miR-34b in Table 5 and the NC mimic sense strand in Table 3 were synthesized, respectively, transfected into 293T cells for 6 h using a LipoFiter™ transfection reagent (Hanbio Biotechnology (Shanghai) Co., Ltd.) together with the SGPL1-3′UTR-WT or SGPL1-3′UTR-MUT plasmid, and then exchanged for a fresh medium to allow transfection for 48 h, and then cells were collected for detection. Successfully transfected cells underwent dual-luciferase assay.

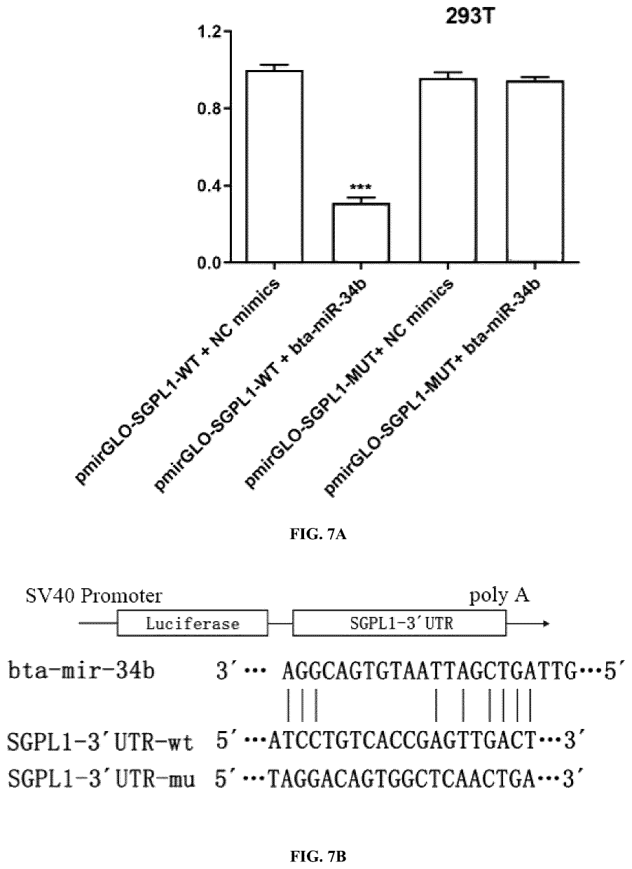

The dual-luciferase assay was conducted using a Promega Dual-Luciferase system kit according to the instructions, and then a Renilla luciferase value was measured and recorded as a luminescence value of the reporter gene. As shown in FIG. 7A: compared with the NC group, bta-miR-34b significantly down-regulated the expression level of luciferase of SGPL1-3′UTR-WT (P<0.001); after mutation, compared with the NC group, bta-miR-34b failed to down-regulate the expression level of luciferase of SGPL1-3′UTR-MUT (P>0.05), indicating that there was a binding effect of the two.

### Example 5 Fluorescence Quantification and Immunofluorescence Detection of SGPL1 Expression Level

Referring to the method in Example 3, the primary cells of Shandong Black cattle were induced to differentiate into preadipocytes, transfected with NC mimic, miR-34b mimic, NC inhibitor, and miR-34b inhibitor separately, continued to allow culturing for 48 h, and then the medium was replaced with a high-glucose DMEM medium with 2% horse serum to continue the culturing.

The cells were collected for RNA extraction and reverse transcribed to obtain cDNA. The cDNA was used as a template to conduct SYBR Green qPCR fluorescence quantification (referring to Example 1) and immunofluorescence assay on SGPL1. The samples in each group were took out and washed 2 times with 1 mL PBS, 3 min each time, and the supernatant was discarded; 1 mL of 4% paraformaldehyde was added to allow fixation at room temperature for 30 min; the samples were washed 2 times with 1 mL PBS, 3 min each time, and the supernatant was discarded; 1 mL 0.5% TritonX-100 was added to allow permeation at room temperature for 30 min; the samples were washed 2 times with 1 mL PBS, 3 min each time, and the supernatant was discarded; 1 mL 5% BSA was added to allow blocking at 37° C. for 1 h, and the blocking solution was discarded; 300 μL PBS-diluted antibody was added to allow incubation at 4° C. overnight; the samples were washed 2 times with 1 mL PBS, 5 min each time, and the PBS was discarded; 300 μL PBS-diluted antibody was added to allow incubation at 37° C. for 1 h; the samples were washed 2 times with 1 mL PBS, 5 min each time, and the PBS was discarded; 300 μL anti-fluorescence quenching mounting solution (containing DAPI) was added; the samples were observed and photographed under a fluorescence microscope.

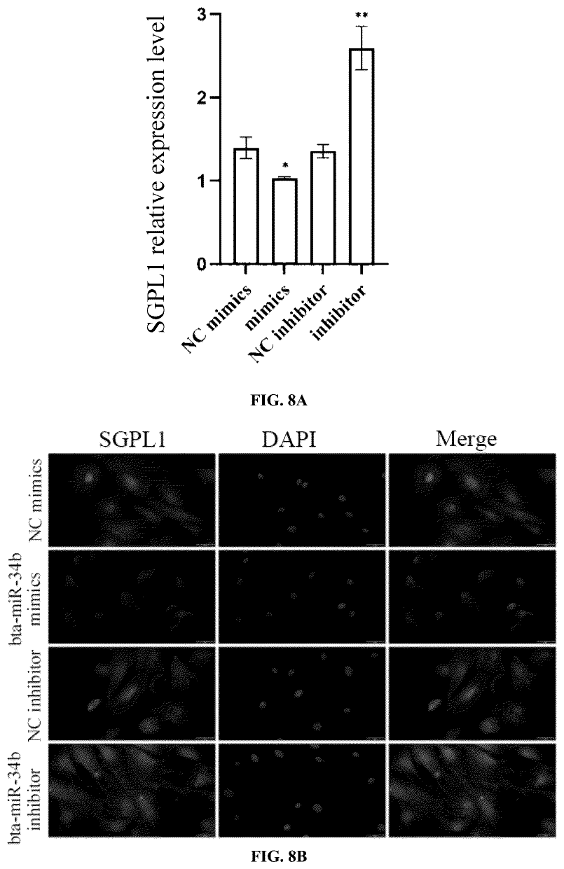

Both the SGPL1 fluorescence quantification (FIG. 8A) and immunofluorescence assay results (FIG. 8B) showed that compared with the NC group, miR-34b mimic inhibited the expression of SGPL1; on the contrary, miR-34b inhibitor promoted the expression of SGPL1.

What is claimed is:

1. A method of breeding a Shandong Black cattle, wherein a miR-34b is used to screen an individual with a low miR-34b level as a stud bull; and
the miR-34b has a nucleotide sequence set forth in SEQ ID NO: 11.

2. A method of regulating adipose proliferation and differentiation of a Shandong Black cattle, wherein a miR-34b is used to transfect a target cell with a vector containing a mimic or an inhibitor of the miR-34b; and the miR-34b has a nucleotide sequence set forth in SEQ ID NO: 11.

3. The method according to claim 2, wherein the mimic of the miR-34b has a nucleotide sequence set forth in SEQ ID NO: 5 or SEQ ID NO: 6; and the inhibitor of the miR-34b has a nucleotide sequence set forth in SEQ ID NO: 9.

4. The method according to claim 2, wherein the vector is selected from the group consisting of a lentivirus, an adenovirus, an adeno-associated virus (AAV), a liposome, and a plasmid for constructing a virus.

5. The method according to claim 2, wherein the target cell is selected from the group consisting of a somatic cell, a fertilized ovum, and a cell line that are derived from the Shandong Black cattle.
